(19) United States
(12) Patent Application Publication
(10) Pub. No.: US 2025/0221655 A1
(43) Pub. Date: Jul. 10, 2025

(54) WEARABLE BIOSIGNAL DEVICE AND SYSTEM FOR INDIVIDUALIZED THERAPEUTIC FEEDBACK

(71) Applicant: IMPULSE WELLNESS LLC, Raleigh, NC (US)

(72) Inventors: Jasper Ian Mark, Raleigh, NC (US); Brady J. Adcock, Raleigh, NC (US); Benjamin Ryle, Raleigh, NC (US); Wallace Shepherd Pitts, Raleigh, NC (US)

(21) Appl. No.: 19/090,889

(22) Filed: Mar. 26, 2025

Related U.S. Application Data
(63) Continuation of application No. PCT/US2023/075239, filed on Sep. 27, 2023.
(60) Provisional application No. 63/377,417, filed on Sep. 28, 2022.

(51) Int. Cl. A61B 5/397; A61B 5/00; A61B 5/256

(52) U.S. Cl. CPC A61B 5/397; A61B 5/0004; A61B 5/256; A61B 5/486

(57) ABSTRACT

The present invention relates to a new apparatus, system, and method for providing therapeutic feedback through use of a biosignal collection device that includes at least one sensor and a monitoring device, such as a computing device, which are communicatively connected to one another preferably in a wireless fashion for measuring, collecting, and analyzing biosignals. In a preferred embodiment, the proposed system features a wearable muscle-sensing device and user-friendly mobile biofeedback application used to improve the daily living of neuro-compromised and motor-compromised individuals. The system can provide users with immediate and real-time feedback on motor recruitment, muscle engagement and coordination, along with a series of customized and therapeutic exercises to enhance their rehabilitative training.

### CROSS-REFERENCE TO RELATED APPLICATIONS

This application is a continuation of PCT Patent Application No. PCT/US2023/075239, titled “WEARABLE BIOSIGNAL DEVICE AND SYSTEM FOR INDIVIDUALIZED THERAPEUTIC FEEDBACK”, filed Sep. 27, 2023, which claims priority to U.S. Provisional Patent Application No. 63/377,417, titled “Biosignal Device for Individualized Therapeutic Feedback”, filed Sep. 28, 2022, the entire contents of all of which are hereby incorporated by reference in their entireties.

### TECHNICAL FIELD

Embodiments of the present invention relate generally to biomedical sensors, and more specifically, to a biosignal detecting garment for measuring a bioelectric signal such as an electromyogram configured to monitor and/or apply therapy to a subject.

### BACKGROUND

Nearly eight million people in the U.S. suffer from stroke, Parkinson's disease, cerebral palsy, and spinal cord injuries; often which result in a survivor's limited ability to control their muscles. Motor-related disorders are often addressed through neuromuscular re-education therapy (NMRT) which has been shown to improve a person's quality of life. Current forms of therapy involve the assessment of electromyography (EMG) signals measured via a plurality of EMG needles/wires or electric adhesive patches adhered on a human body. More specifically, these electric adhesive patches are adhered on different portions of the human body to sense the EMG signals and are electrically connected to a monitor to analyze the signals for displaying. These signals are then interpreted by a medical specialist.

For added convenience, these electric adhesive patches have been integrated with a wearable garment such that the electric adhesive patches are adhered on the garment and in close contact with the skin of the human body. However, the electric adhesive patches will lose their adhesive or become folded in such a manner that the physiological signals cannot be measured or are not reliable.

The current state of the art includes EMG systems that often collect biosignals through needles inserted invasively through the skin and into muscles or conventional wet silver chloride (AgCl) electrodes placed over the muscle. These systems have a number of drawbacks, however. First, the AgCl electrodes require users to have a foundational understanding of the musculoskeletal system to ensure electrodes are accurately placed and signals are valid, reproducible, and repeatable. Second, these systems fail to provide meaningful insights into muscle synergies or co-contractions that arise during task-based movements. Third, these systems fail to provide patients and everyday users with meaningful information and feedback, as these systems are not designed for the target audience of individuals who have suffered a traumatic motor-deficit inducing injury.

In view of the foregoing, it would be advantageous to provide wearable surface sensors or electrodes that are convenient for use, do not require traditional electric adhesive patches, and provide reliable results. It would be further advantageous to provide a wearable biosignal device that can collect and transmit signals to a computing device, whereby the signals are interpreted and displayed in a way that is meaningful and understandable to a layperson. Furthermore, it would be beneficial for the displayed results and feedback to include specific customized and therapeutic exercises and rehabilitative muscular training for the user.

### SUMMARY

This summary is provided to introduce in a simplified form concepts that are further described in the following detailed descriptions. This summary is not intended to identify key features or essential features of the claimed subject matter, nor is it to be construed as limiting the scope of the claimed subject matter.

Embodiments described herein relate to a new apparatus, system, and method for providing therapeutic feedback through use of a biosignal collection device that includes at least one sensor and a monitoring device, such as a computing device, which are communicatively connected to one another preferably in a wireless fashion for measuring, collecting, and analyzing biosignals such as an electroencephalogram, an electromyogram, and an electrocardiogram. In a preferred embodiment, the proposed system features a wearable muscle-sensing device and user-friendly biofeedback application used to improve the daily living of neuro-compromised and motor-compromised individuals. The system can provide users with immediate and real-time (or near real-time) feedback on motor recruitment, muscle engagement and coordination, along with a series of customized and therapeutic exercises to enhance their rehabilitative training. The embodiments described herein provide a system that is capable of encouraging compliance along with engagement. Such components are utilized to mitigate the key factors that can hinder progress of a rehabilitation program.

An object of the present invention is to provide a therapeutic system that includes a biosignal device that is wearable, intuitive to use, and serves as a therapeutic system for the user. The system may include at least one sensor, or a plurality of sensors, embedded into a wearable textile, whereby the sensor(s) transmit collected biosignals to a communication terminal wherein the signals are pre-processed and further transmitted to a computing device. The biosignal data is transmitted to a communication terminal comprising a circuit assembly that may be sheathed in an electronic housing on the wearable device, wherein the biosignal data is processed and then transmitted to the computing device that may be external to the wearable biosignal device. The computing device includes a processor that further processes the data received from the circuit assembly into information that is readable by the user and displays that information for the user. The displayed information is then used to provide meaningful feedback that can be interpreted by the user without the assistance or interpretation of a medical professional or one trained in biosignal readings. Moreover, the computing device analyzes the data and provides therapeutic action-based games for the user to encourage a user's engagement in a therapeutic and rehabilitative process.

In one embodiment, the system includes at least one reusable sensor worn by a user against the user's skin, whereby the sensor(s) is/are in contact with the user's skin and measures and collects biosignals from the user. The sensor(s) transmits the collected biosignals wirelessly to a computing device. The sensor(s) do not require the use of tape or any other adhesive components to maintain proper skin-sensor site connection. The sensor(s) are integrated and/or embedded into or onto fabric or other textile-based materials, creating a wearable biosignal device (also referred to herein as “wearable device”). The sensor(s) may be arranged in various combinations, including but not limited to, monopolar, bipolar, or a multiple of such. Such biosignals may be measured with or without a reference/ground sensor. The biosignal device may also include sensors that are capable of collecting information regarding a user's rate of respiration as well as positional and kinematic data such as acceleration, velocity, and perceived force data.

In a preferred embodiment, the sensor(s) may be used to collect electromyography (EMG) signals and can be embedded in a material such as a textile fabric in a manner such that the sensor is secured within the fabric while keeping the contact surface of the sensor sufficiently exposed from the fabric to make contact with a user's skin when placed on the user. In this embodiment, the wearable biosignal device may be summarized as including at least one electromyography (EMG) sensor responsive to muscle activity corresponding to a gesture performed by a user of the wearable biosignal device and to provide signals in response thereto, a communication terminal, comprising a circuit assembly, communicatively coupled to the sensor(s), and a computing device communicatively coupled to the communication terminal. The computing device stores processor-executable instructions that, when executed by the processor, cause the processor to identify, in real-time or near real-time, a gesture performed by the user based at least in part on signals provided by the sensor(s). Furthermore, the computing device analyzes biometric data using a plurality of calculations and measurements described herein.

The wearable biosignal device includes a set of communication pathways carried by the wearable device and a set of components carried by the wearable device. The set of components may include at least one sensor, such as an electrode or other contact sensor, and at least one communication terminal, comprising a circuit assembly, wherein the communication terminal is communicatively coupled to the sensor(s) by at least one communication pathway in the set of communication pathways. The communication terminal may include at least one of a wireless communication terminal and/or a wired communication terminal. Communicative coupling between the sensor(s) and the communication terminal may be mediated by at least one additional component in the set of components carried by the device.

The term “communicative” as in “communicative pathway,” “communicative coupling,” and in variants such as “communicatively coupled,” is generally used to refer to any engineered arrangement for transferring and/or exchanging information. Exemplary communicative pathways include, but are not limited to, electrically conductive pathways (e.g., electrically conductive wires, electrically conductive traces), magnetic pathways (e.g., magnetic media), and/or optical pathways (e.g., optical fiber), and exemplary communicative couplings include, but are not limited to, electrical couplings, magnetic couplings, and/or optical couplings. Furthermore, the term “communicatively coupled” is generally used to include direct, 1:1 communicative coupling and indirect or mediated communicative coupling. For example, a component A may be communicatively coupled to a component B directly by at least one communication pathway, or a component A may be communicatively coupled to a component B indirectly by at least a first communication pathway that directly couples component A to a component C and at least a second communication pathway that directly couples component C to component B. In this case, component C is said to mediate the communicative coupling between component A and component B.

The textile portion of the wearable device may be fashioned in the form of a fabric sheet comprising a first surface (or outer surface) that can be worn outward with respect to a user's body and a second surface (or inner surface) that can be worn inward with respect to a user's body. In one embodiment, the textile portion can be applied circumferentially around a portion of the user's body such that the contact surface of the sensor is exposed on the inner surface of the sheet and placed directly on the skin, making contact with the skin sufficiently to measure and/or record a biosignal. The wearable device may be secured around the user by means of a fastener such as hook and loop, snap button, and the like. Alternatively, the wearable device and material may be manufactured initially in the form of a circumferential band that may be slid around a portion of the user's body, such as an arm or leg. In addition to measuring and collecting biosignals produced by the user, the sensor(s) can pass signals through the body. These signals may be used to assess skin impedance, whereby a high frequency current is passed through the body via a single sensor A+, for example, to be received through any sensor across the body such as A−, B+, B−, and so on, for example, thus determining if the sensor(s) are making adequate contact with the skin, or if they have lost their conductive ability and need to be replaced.

Following application of the wearable device to a user's body, the sensor measures and collects biosignals produced by the user or the user's environment and transmits the collected signals to a communication terminal that may be comprised in an electronic housing preferably incorporated into the wearable device by means such as insertion into a sleeve that may be affixed to the fabric, an elastic band that may be secured around the housing, or other such means of attachment. The communication terminal collects, amplifies, and/or filters the received biosignals for further transmission to a computing device, either wirelessly or via a direct analogue connection. This may include, but is not limited to, transmission over Bluetooth, Bluetooth Low Energy (BLE), or an auxiliary cable connection. Alternatively, digital signal processing may be performed within the computing device. The processed data is used to modulate an onscreen display, where modulation is a direct result of the signal, or interaction of signals, collected. In yet another embodiment, positive feedback can be provided in the form of motivational feedback and encouragement. In still another embodiment, individualized feedback will be provided as determined by an overall muscle communication and health score, where values are calculated by including several EMG-based biomarkers which may include, but is not limited to, a muscle ratio, intermuscular coherence, or muscular power/signal strength.

### BRIEF DESCRIPTION OF THE DRAWINGS

The foregoing, as well as the following Detailed Description, is better understood when read in conjunction with the appended drawings. For the purposes of illustration, there are shown in the drawings exemplary embodiments; however, the presently disclosed subject matter is not limited to the specific methods and instrumentalities disclosed. These and other features, aspects, and advantages of the present invention will become better understood with regard to the following description, appended claims, and accompanying drawings where:

FIG. 1 is a perspective view of an embodiment of the wearable biosignal device worn on an anterior portion of a user's arm.

FIG. 2 is a perspective view of an embodiment of the wearable biosignal device worn on a posterior portion of a user's arm.

FIG. 3 is a top view of an exemplary arrangement of a sensor and its connection to the communication terminal.

FIG. 4 is a cross-sectional view of an embodiment of a sensor and its connection to the communication terminal embedded into the wearable portion of the biosignal device.

FIG. 5A is a block diagram of an exemplary embodiment of the communication terminal circuitry of the wearable biosignal device.

FIG. 5B is a block diagram of an exemplary embodiment of detailed components and functions of the communication terminal circuitry of the wearable biosignal device.

FIG. 6 is a flow chart of an exemplary embodiment of the general operating environment system.

FIG. 7 is a flow chart of an exemplary embodiment of a user interaction with the wearable biosignal device and system.

FIG. 8 is a graph of raw and processed electromyography signals.

FIG. 9 shows an exemplary embodiment of an operating environment with a viewable representation of data presented on a computing device following user modulation of the wearable biosignal device.

FIG. 10 depicts a block diagram illustrating one embodiment of a computing device in accordance with the subject matter described herein.

### DETAILED DESCRIPTION

The following description and figures are illustrative and are not to be construed as limiting. Numerous specific details are described to provide a thorough understanding of the disclosure. In certain instances, however, well-known or conventional details are not described in order to avoid obscuring the description. Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that same thing can be said in more than one way.

Alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

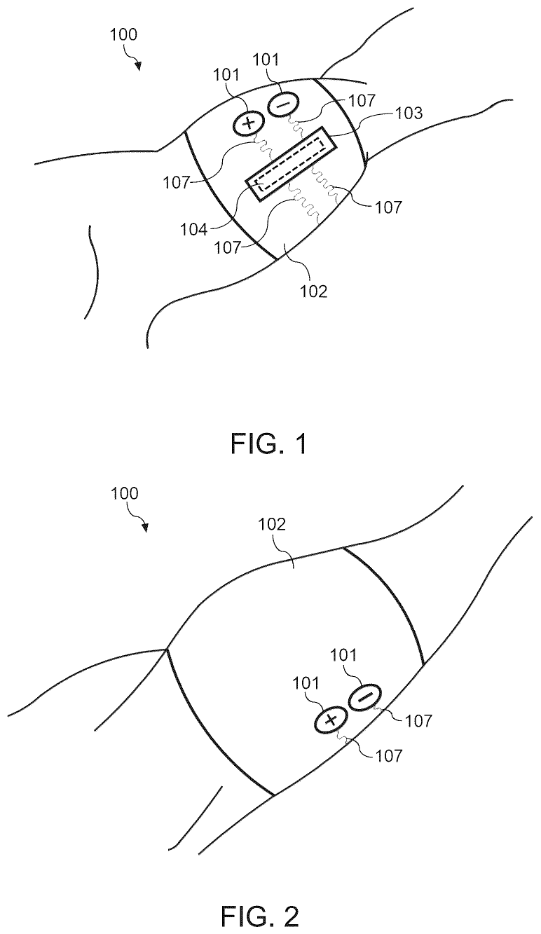

FIG. 1 and FIG. 2 show an anterior and posterior view, respectively, of one embodiment of the wearable biosignal device 100 worn circumferentially on a user's arm with a bipolar sensor 101 configuration for bipolar biosignal collection. It is to be understood that the number of sensors 101 can vary depending on the optimal number needed for biosignal collection; and indeed, it is an advantage of the present system to have the ability to incorporate a plurality of sensors 101 on a single wearable device 100 thus allowing for the measurement and collection of a multitude of biosignals simultaneously. Sensors 101 may include any type or types of contact sensors, including without limitation one or more EMG sensor(s), one or more magnetomyography sensor(s), one or more acoustic myography sensor(s), one or more mechanomyography sensor(s), one or more electrocardiography sensor(s), one or more blood pressure sensor(s), one or more thermometer(s), and/or one or more skin conductance sensor(s). Contact sensors may include any type or types of biometric sensor(s) that are responsive to signals detected through physical contact with the user's skin. The wearable biosignal device 100 may, if desired, also include one or more other form(s) of sensor(s) 101, such as one or more pedometer(s), one or more inertial sensor(s) such as one or more accelerometer(s) and/or one or more gyroscope(s), one or more altimeter(s), and so on.

In FIG. 1, positive sensor 101 (shown with a “+”) and negative sensor 101 (shown with a “−”) are embedded in a fabric or textile band 102 having an inner surface and an outer surface. The positive sensor 101 and the negative sensor 101 form a sensor coupling or a sensor pair. The embedding in the fabric or textile band may be achieved through the manner of weaving the sensor(s) 101 into the band 102, for example. The textile band 102 may comprise a single piece of material (e.g., elastic material, flexible material, stretchable material, etc.) or multiple sections of material adaptively coupled together permitting limited motion of the sections with relation to one another. The textile band 102 may be substantially planar when laid out flat but may generally be curved in use. Moreover, the textile portion 102 of the device 100 may be fashioned into any appropriate shape in addition to a band, such as a form fitting shirt or bodysuit, thus allowing for a plurality of muscle-groups (in the case of electromyography) to be analyzed at a single time.

From this perspective view, the sensors 101 are embedded in a position such that the top surface of the sensors 101 are visible on the outer surface of the textile band 102. It is to be appreciated that the top surface of the sensors 101 may or may not be visible on the outer surface of the textile band 102, provided that the contact surface of the sensor 101 is exposed on the inner surface of the textile band 102 such that the sensor 101 contact surface can make sufficient skin-sensor contact to measure and/or collect a biosignal. The positive sensor 101 and the negative sensor 101 make up a sensor 101 coupling for an individual muscle or muscle groups on the anterior side of a user's arm in this arrangement, and the sensors 101 are communicatively connected to an electronic housing 103. The electronic housing 103 contains a communication terminal 104 comprising a circuit assembly 105 capable of receiving, processing, and transmitting biosignals measured or collected via the embedded sensors 101 to a computing device 106. The exterior of the electronic housing 103 may feature controls, including on/off power switching and Bluetooth pairing.

FIG. 2 depicts an embodiment of the biosignal device 100 in the same assembled state as FIG. 1 from a posterior view of a user's arm. The positive sensor 101 (shown as “+”) and negative sensor 101 (shown as “−”) are also communicatively connected to the communication terminal 104 via wire interconnects 107 at an attachment point of the electronic housing 103. The positive sensor 101 and the negative sensor 101 shown in FIG. 2 form a different sensor coupling or sensor pair than the positive sensor 101 and the negative sensor 101 shown in FIG. 1. Alternatively, the communication terminal 104 may be communicatively coupled directly to the sensor 101 whereby the collected biosignals may be transmitted wirelessly from the sensor 101 to the communication terminal 104. The wearable device 100 placement of this illustrated embodiment can assess two muscles (or muscle groups) via bipolar signal acquisition. The process of this signal collection, transmission, and assessment will be discussed in greater detail herein with reference to additional figures.

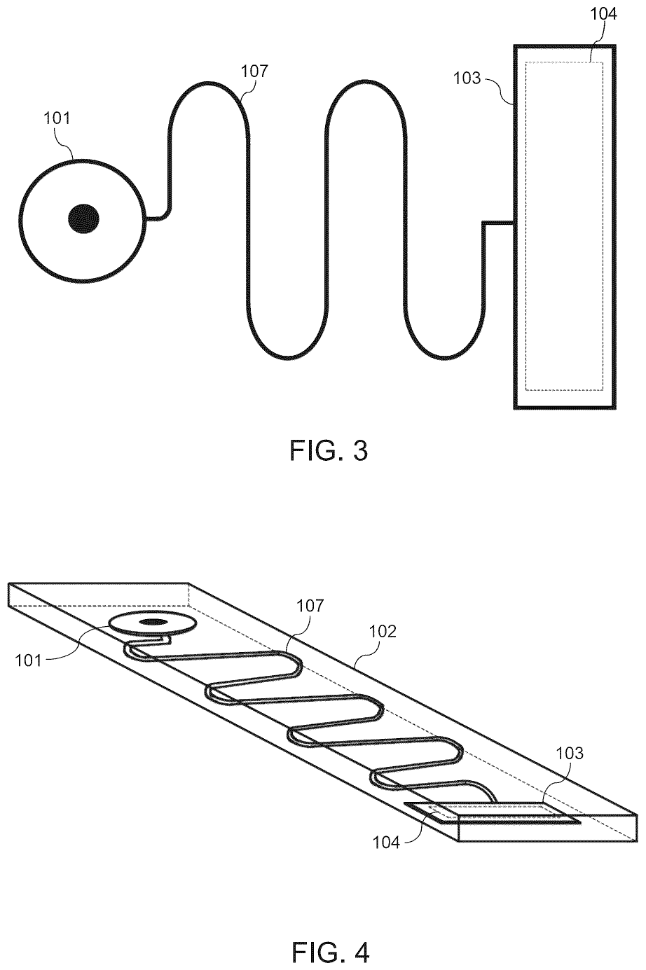

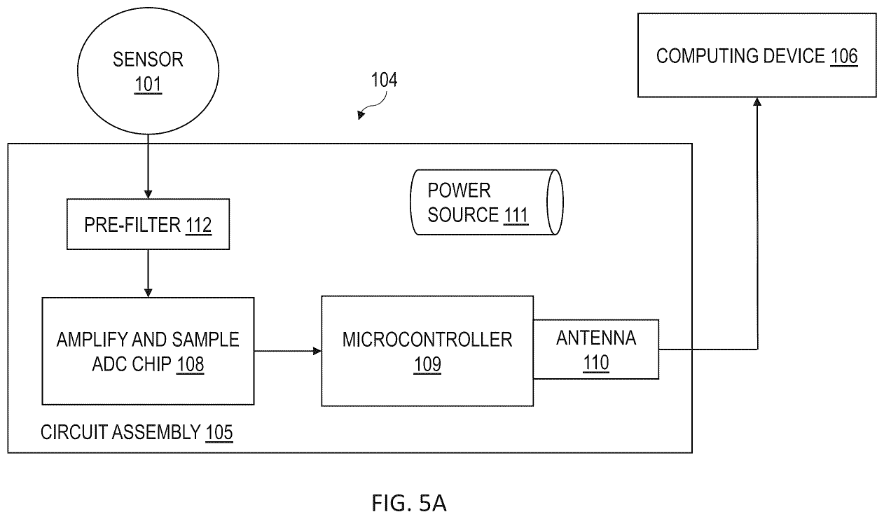

An exemplary arrangement of a sensor 101 and its connection to the communication terminal 104 sheathed in the electronic housing 103 is illustrated in FIG. 3. The sensor 101, which may relate to a positive or negative terminal, transmits conducted biosignals along a wire interconnect 107 to the circuit assembly 105 of the communication terminal 104 sheathed within the electronic housing 103. FIG. 4 shows a cross-sectional view of a portion of the wearable device 100 comprising the sensor 101, a wire interconnect 107, and the communication terminal 104 within the electronic housing 103. FIG. 4 demonstrates the positioning of FIG. 3 with respect to being embedded within a textile fabric band 102. In FIG. 4, the upper surface of the textile fabric 102 is cut away so that the arrangement of the sensor 101 connected to the wire interconnect 107 and connection of the wire interconnect 107 to the communication terminal 104 can be more easily illustrated. In this cross-section, the top surface of the sensor 101 can be seen through the upper surface of the textile band 102, and the contact surface of the sensor 101 is positioned within the inner portion of the textile band 102 to make contact with the user's skin (not shown).

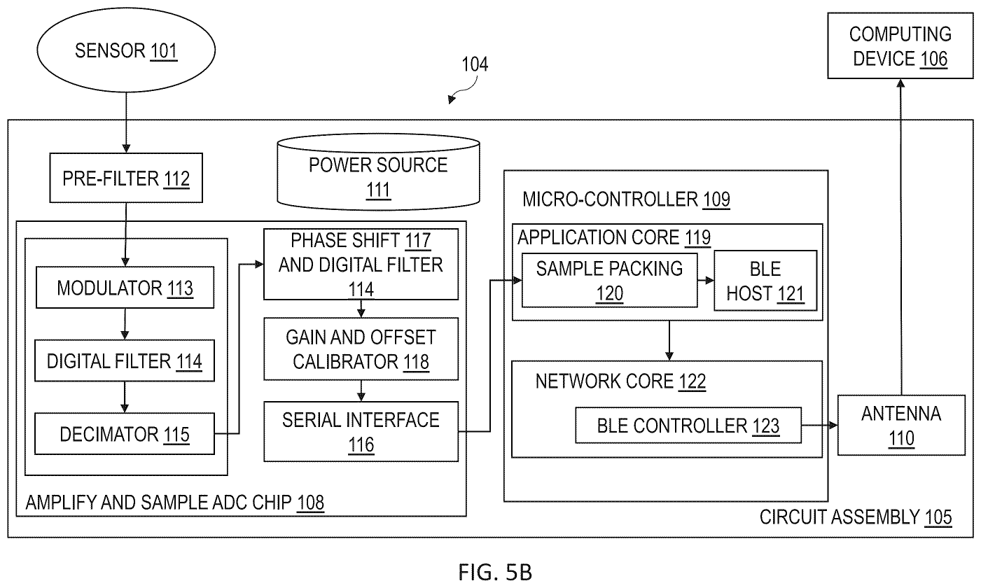

FIG. 5A is a block diagram of an exemplary embodiment of the circuit assembly 105 of the communication terminal 104 of the biosignal device 100. It is to be understood that different processing components and steps may be employed to achieve signal processing and transmission, and the process and components described herein exemplify a preferred embodiment. The exemplary circuit assembly 105 in FIG. 5A is circuitry (e.g., electrical and/or electronic circuitry) that is communicatively coupled to sensors 101 and may include a wide variety of components depending on the specific implementation. The circuit assembly 105 includes an amplification circuit to amplify signals provided by sensors 101, a filtering circuit to filter signals provided by sensors 101, an analog-to-digital converter to convert analog signals provided by sensors 101 into digital signals, and a digital processor to process the signals provided by sensors 101. In this preferred embodiment, an ADC chip 108 amplifies and samples the biosignals in a single chip, and a microcontroller 109 further processes the signals to be transmitted to the computing device 106. The microcontroller 109 prepares the signal data to be transmitted wirelessly via antenna 110 to the computing device 106 to store processor-executable instructions that, when executed by the digital processor, cause the digital processor to process the signals provided by the sensor(s) 101. In other implementations, the circuit assembly 105 may include without limitation: one or more power sources 111, and/or one or more communication terminal(s) 104 such as one or more wireless transmitter(s) and/or receiver(s) (either separately or combined as a wireless transceiver) employing a wireless communication protocol such as Bluetooth®, WiFi™, and/or NFC™, one or more tethered connector port(s) (e.g., one or more Universal Serial Bus (USB) port(s), one or more mini-USB port(s), one or more micro-USB port(s), and/or one or more Thunderbolt® port(s)), and/or any other form or forms of communication terminal(s) 104.

Throughout this specification and the appended claims, the term “communication terminal” is generally used to refer to any physical structure that provides a communications link through which a data signal may enter and/or leave a device or a component of a device, such as the wearable biosignal device 100 of the present invention. A communication terminal represents the end (or “terminus”) of communicative signal transfer within a device (or a component of a device) and the beginning of communicative signal transfer with an external device (or a separate component of the device). In the case of a communication terminal 104 in the circuit assembly 105, the term “terminal” means that the communication terminal 104 in the circuit assembly 105 represents the end of communicative signal transfer within the wearable biosignal device 100 and the beginning of communicative signal transfer with other components of the biosignal device 100 and/or with one or more device(s) separate from the wearable portion of the device (e.g., a computing device 106 such as one or more smartphone(s), one or more desktop, laptop, or tablet computer(s), etc.).

A more detailed description of a preferred embodiment of the communication terminal 104 circuit assembly 105 of FIG. 5A is shown in FIG. 5B. The circuit assembly 105 comprises a power source 111 such as a battery, a pre-filter module 112, two chip packages 108 and 109, and an antenna 110. In this embodiment, the power source 111 is provided by a portable voltage supply such as a battery that may be connected to a buck boost converter, thereby producing a steady and constant voltage supply such as 3.3 volts or 5 volts. The signals pass through a pre-filter 112 comprised of synergistic resistors and capacitors assembled in series and in parallel, wherein the signals are filtered according to an algorithm suitable to the type of diagnostic test or signal collection that is being performed. For instance, noise reduction algorithms may be applied to reduce noise and improve signal to noise ratio. Additionally, pattern recognition or other detection algorithms may be applied to the signals to optimize signal frequencies. These algorithms are commonly known to those skilled in the art. In this embodiment, the pre-filter 112 is a band-pass filter set to a frequency range of 0.05 Hz to 20,000 Hz.

Filtered analog signals are amplified, sampled, and converted to digital signals by an analog-to-digital converter (ADC) chip 108. In current medical instrumentation amplifiers and ADCs, conventional approaches typically involve separate components or multiple chip packages for signal acquisition, amplification, and digital conversion. In these conventional systems, an analog biosignal or medical signal from a sensor is transmitted to a series of amplifiers for signal magnification and then sent to a separate ADC for conversion into a digital signal. This process introduces potential signal degradation, noise interference, increased complexity, and a larger system footprint. The method of amplification described herein overcomes the limitations of conventional systems and provides a combined amplify and sample analog-to-digital conversion within a single-chip package, enabling direct amplification and sampling of analog biosignals or medical signals without the need for external components or interconnections.

The ADC chip 108 has a built-in functionality to control gain, sample rate, error checking, etc. by configuring control registers (or memory) of the ADC chip 108 that define the amplification factor, sample rate, etc. Custom firmware is flashed onto the microprocessor that controls the ADC chip 108, allowing for a dynamic setting of the registers of the ADC chip 108 based on the needs of the application, thus allowing for the augmentation of the gain, sample rate, and other functionalities of the ACD chip 108. The drivers are customized to direct and handle the data as it comes across the serial interface 116 of the ADC chip 108. These customized drivers communicate with the ADC chip 108, store the incoming samples into various packets, and send them over BLE without delay. The drivers utilize nRF software development kit (SDK) libraries in a communication pathway over a serial peripheral interface (SPI), an interface bus used to send data between microcontrollers and peripherals (in this case, the ADC chip 108). The drivers are used to regulate clock-speeds, change sample rates, synchronize data, and ensure consistency in packaging data for BLE transfer regardless of sample rate and without delay. Additionally, the driver enables power saving as well as error checking of samples and is also able to compress the data before sending.

A modulator 113 determines at what point in the time domain the signal will be sampled, samples the signal, then converts the signal from analog to digital. A digital filter 114 and decimator 115 filters to a frequency range of 0.05 Hz to 20,000 Hz and reduces the number of sampled inputs to match a requested sample rate or clock rate set by the microcontroller 109. For instance, if data is coming in at a speed of 64 kHz from the biosignal sensor 101, and the speed programmed at the serial interface 116 to be read by the microcontroller 109 is 8 kHz, the data will be decimated accordingly. The phase shifter 117 and digital filter 114 realigns the frequency wave to a continuous data stream to eliminate any delay in wavelet phase and can be programmed by the user on the computing device 106 as determined by the capability of the user-end computing device 106. The gain and offset calibrator 118 amplify the signal to reach the wireless signal transmission threshold and, if necessary, will perform a voltage offset to regulate the signal. At the point of serial interface 116, the preprocessed data is transmitted from the ADC chip 108 to the microcontroller 109 comprising two cores: an application core 119 comprising a BLE host 121 wherein sample packing 120 occurs preferably at 24-bit but can be lowered for efficiency (such as 16-bit), and a network core 122 controlling the wireless signal transmission radio hardware, such as a BLE controller 123, whereby the signals are sent wirelessly via an antenna 110 to a computing device 106. The computing device 106 performs advanced signal processing and calculations such as intermuscular coherence, muscle activation ratio, co-contraction index, target-amplitude precision and accuracy, mean power frequency, average amplitude, signal envelope, data averaging, Fourier transforms, root mean square, signal burst, recruitment slope, peak frequency, and smoothed signal. These methods are discussed in further detail herein.

Depending on the type of medical test being conducted, such as electroencephalogram (EEG), electromyography (EMG), or electrocardiogram (EKG) for example, the physiological signals will have unique signal characteristics, and signal processing steps can be adjusted for the particular signal being collected. The software on the computing device 106 allows for an adjustment of the frequency of interest whereby the microcontroller 109 communicates to the decimator 115 the appropriate sample rate as determined by the desired frequency range.

During an EEG, measurement of the brain's electrical activity is typically broken up into 5 frequency bands: delta (0.5-4 Hz), theta (4-8 Hz), alpha (8-13 Hz), beta (13-30 Hz), and gamma (30-100 Hz). Each is associated with unique body states, and time-frequency analysis is performed, including wavelet transformation or via a FFT to extract this information. EEG voltages range typically from 0-200 μV, and power analysis can be performed in the voltage domain as well to explore brain activity during various processes.

EMG is a measurement of the muscle's electrical activity, and this is processed in a similar function to EEG data above. EMG, however, has a wider frequency bin, typically from 10-500 Hz. And although the device 100 described herein preferably focuses on 0.5-500 Hz, the device 100 is capable of collecting frequencies from 0.001-64,000 Hz. As with an EEG, various time-frequency analysis or voltage-domain analysis (power analysis) can be performed. Voltage range for EMG is typically between 0.05 to 10 mV. EMG analysis can compute measures reflecting fatigue, muscle communication, or healthy movement.

When measuring heart electrical activity in an EKG, distinct frequency analysis is not always necessary, although frequencies typically range from 0-20 Hz. Instead, it is preferable to focus on wave characteristics such as the P wave (or atrial depolarization), QRS complex (or ventricular depolarization), and T wave (or ventricular repolarization). Voltage domain measurements are more crucial, whereby the P wave is in the 0.1-0.25 mV range, the QRS complex is 0.5-1.5 mV, and the T wave is 0.1-0.5 mV.

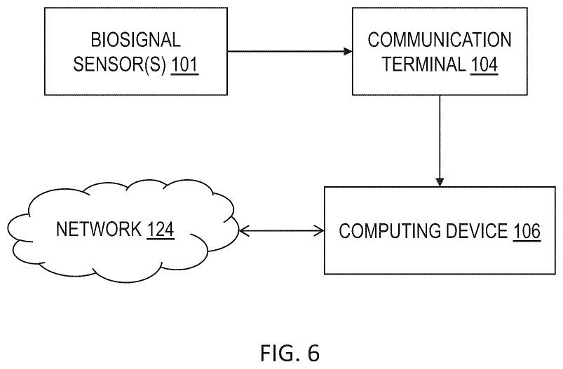

A simplified overview of the system operating environment is illustrated by the flow chart of FIG. 6. The flow chart illustrates the flow of data gathered by sensors 101 attached to a user wearing the biosignal device 100. The data is transmitted to the communication terminal 104 for interpretation and signal processing and then further transmitted to a computing device 106 for additional processing, display, and local user feedback. The computing device 106 may communicate on an open network 124 for exchanging user information and pushing new interaction experiences to the end user. Furthermore, any data collected by the computing device 106 can be stored on the device 106 itself or in cloud storage for retrieval.

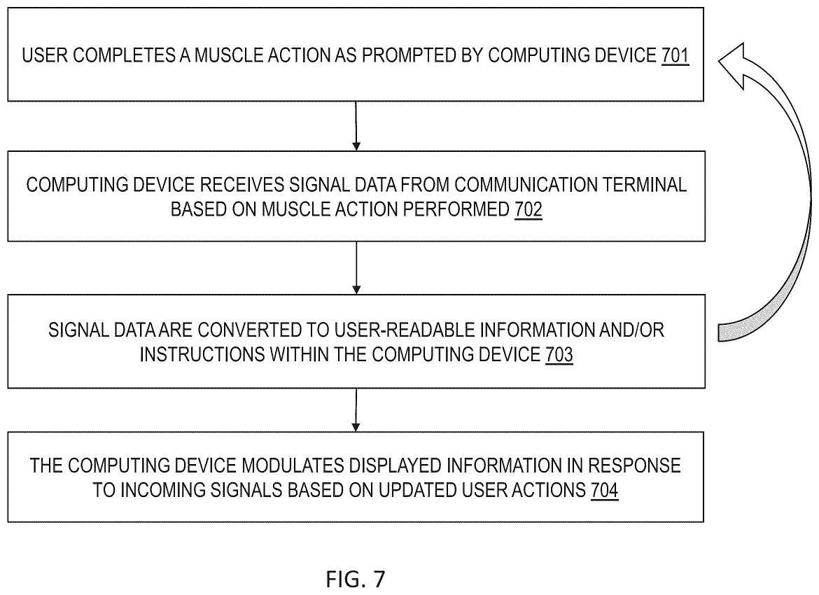

FIG. 7 is a flow chart illustrating the workflow of a computing device 106 and biosignal interaction in an embodiment where the sensors 101 of the wearable biosignal device 100 are EMG sensors for detecting and processing the electrical signals generated by muscle activity. In this embodiment, the wearable device 100 employs EMG sensors 101 that are responsive to the range of electrical potentials (typically μV-mV) involved in muscle activity. This exemplary operating environment features first step 701, whereby a user completes a muscle-based action item prompted by the computing device 106 while wearing the biosignal device 100. The wearable device 100 detects muscle activity corresponding to a physical gesture performed by the user. The wearable device 100 processes EMG signals corresponding to the detected muscle activity. The gesture or action performed may be any movement of the body or joints, along with an isometric contraction of the body. Measurements may also be taken while the user is at rest to determine if involuntary muscle movement occurs when a user is not intentionally moving, such as a muscle spasm. In a second step 702 of FIG. 7, the biosignal data collected and measured from the EMG sensor 101 (or array of sensors 101) are transmitted from the communication terminal 104 to the computing device 106 based on the action performed. During conventional EMGs, the biosignals are translated into graphs or numbers that are typically read by a medical practitioner or a professional trained in biometric readings; however, the present therapeutic system overcomes some of the disadvantages of conventional EMGs by displaying a viewable representation 127 of biometric data that is user-friendly and by providing therapeutic exercises and interactive games as a form of rehabilitation. As shown in a third step 703 of FIG. 7, the computing device 106 of the present wearable device 100 is configured to convert the signals received from the communication terminal 104 of the wearable device 100 to a viewable representation 127 of information and feedback that is interpretable by the user. This information and feedback can be displayed to the user in the form of onscreen text, audio, graphics, or any other means of illustrating the converted data. The viewable representation 127 of data conveyed to the user may be qualitative or quantitative indicating the health of the muscles and the motor neurons that control them. For example, individualized scores can be assigned to a specific muscle or muscle group indicating the health and function of that muscle group when compared to a baseline or control group.

Furthermore, the software can suggest a target action, such as a rehabilitative exercise or series of exercises, within the computing device 106. In a fourth step 704 shown in FIG. 7, the computing device 106 of the therapeutic system modulates the information displayed to the user in response to newly acquired incoming signals based on updated user actions. In other words, the target action items are updated with respect to previously completed actions, and new action item recommendations are communicated to the user in real-time (or near real-time), thus creating a feedback loop that provides timely and relevant information 127 reflecting the user's activity.

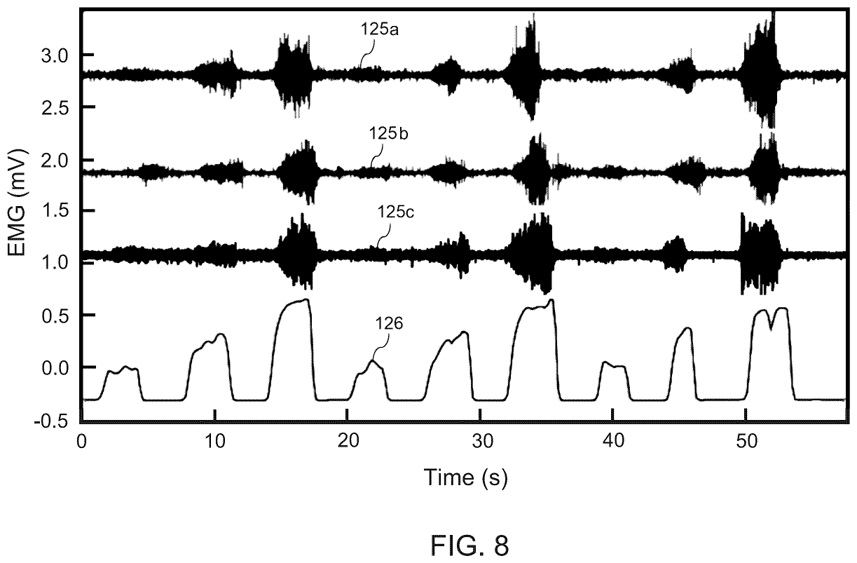

The biosignal data transmitted from the communication terminal 104 of the wearable device 100 to the computing device 106 is pre-processed raw EMG data 125 that is further processed and smoothed by the computing device 106 to display a more streamlined volume or signal 126 to the user. FIG. 8 shows an example of raw EMG biosignal data 125a, 125b, and 125c in the form of three separate raw EMG channels. The three separate raw EMG channels may each represent signals gathered from different sensors or sensor pairs. Once the signal is filtered, the processed signal 126 displays data that is smoother and easier to interpret. The computing device 106, using one or more hardware processors, analyzes amplitudes and time frequencies and runs a discrete Fourier transform on the incoming biosignal(s). The biosignal(s) is also broken up into the different frequency bands of interest. In the case of an EMG, the computing device 106 can compute an intermuscular coherence to analyze the incoming biosignal(s) from a plurality of sensors 101 to determine the similarity in signal and to assess how individual muscles are working together to perform a specific action. A muscle ratio is also computed that analyzes agonist/antagonist interactions between specific muscles or muscle groups. For example, in FIG. 8, a smaller amplitude corresponds to a muscle squeezing moderately, whereas a larger amplitude corresponds to a muscle squeezing harder.

Collected biosignals are compared with relation to predefined parameters such as predefined target frequency and voltage parameters as well as other biosignal characteristics. For example, if a user is instructed to squeeze a muscle to fifty percent of their maximum, the user's waveform amplitude can be compared to a target amplitude to identify the delta between the maximum and what the user is able to sustain. In another example, the computing device 106 can analyze transmitted biosignal data for signs of muscle fatigue. Certain frequency characteristics are associated with muscle fatigue, and the computing device 106 can analyze the biosignal data for certain waveform shifts to determine if a user is fatiguing earlier or later than a target value. If a user is fatiguing earlier or later than the predefined target value, the computing device 106 can encourage the user to either reduce the muscular burden or reduce the muscular effort thereby sustaining a more effective therapy protocol.

The present therapeutic system can use a plurality of sensors 101 to assess individual muscles within a specific muscle group for analysis of the muscle function and their interaction with respect to the muscle group. For example, current methods of collecting EMG signals may employ one large sensor that assesses a muscle group, such as the quadriceps; however, the present system can incorporate four smaller sensors 101 on the wearable device 100, each sensor 101 positioned to measure each of the four quadricep muscles within the quadricep muscle group, thereby assessing all four quadriceps individually with respect to one another within the quadricep group. In this example, the biosignals of the four muscles are sent simultaneously yet separately in parallel. Each quadricep muscle will produce a unique waveform (such as in FIG. 8) that is transmitted from the sensor 101 to the communication terminal 104 of the wearable device 100. The computing device 106 receives the biosignal data from the communication terminal 104 and runs an independent component analysis (ICA) using an artificial intelligence algorithm that extracts and separates the wavelengths, thereby determining which electrode the wavelength is attributed to within the wearable device 100.

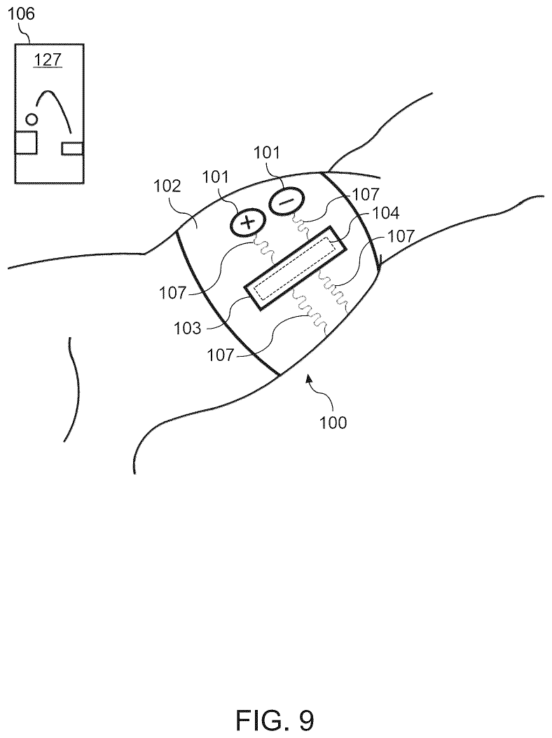

The processing methods performed by the computing device 106 and described herein are utilized in the programming of therapeutic games offered on computing device 106 of the therapeutic system. FIG. 9 is a graphical depiction of the therapeutic system operating environment whereby a muscular rehabilitative game is displayed on the computing device 106. In other words, the viewable representation 127 of the biometric data is presented to the user in the form of a game. Following the muscle-based action of the user, the computing device 106 displays actions coinciding with user movement. For example, if the user is playing a lumberjack game on the computing device 106, the flexing of a bicep muscle will cause the lumber jack to swing an axe. The harder the user squeezes their bicep, the faster the lumberjack will swing the axe. The games provided by this system encourage user engagement and compliance with rehabilitative therapies.

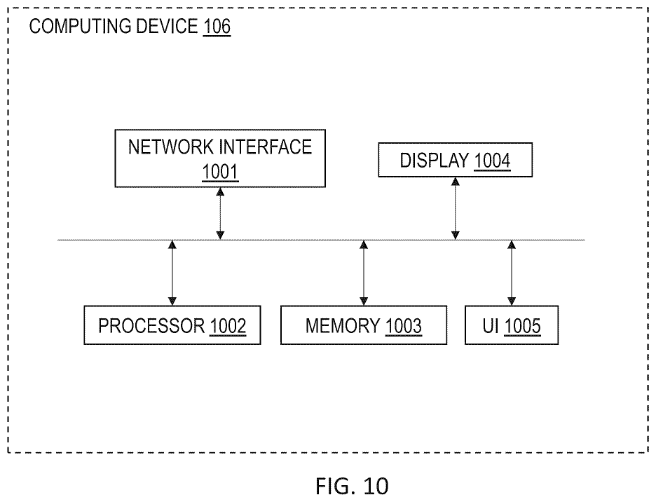

FIG. 10 depicts a block diagram illustrating one embodiment of a computing device 106. Referring to FIG. 10, the computing device may be computing device 106 referred to in FIG. 5A, FIG. 5B, and FIG. 6, for example. The computing device 106 may include network interface 1001, at least one processor 1002, a memory 1003, a display 1004, and a UI 1005. The memory 1003 may be partially integrated with the processor 1002. The UI 1005 may include a keyboard and a mouse. The display 1004 and the UI 1005 may provide any of the GUIs in the embodiments of this disclosure. The computing device 106 may be a mobile device, such as a smart phone or a smart tablet that further includes a camera, WAN radios, and LAN radios. In some embodiments, the mobile device may be a laptop, a tablet, or the like. The incoming signals described herein may be received over network interface 1001 and processed by the one or more processor 1002 and displayed on display 1004 via one or more UI 1005, and the signals may further be saved to memory 1003.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Spatially relative terms, such as “below,” “beneath,” “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation, in addition to the orientation depicted in the figures. Throughout the specification, like reference numerals in the drawings denote like elements.

Embodiments of the inventive subject matter are described herein with reference to plan and perspective illustrations that are schematic illustrations of idealized embodiments of the inventive subject matter. As such, variations from the shapes of the illustrations as a result, for example, of manufacturing techniques and/or tolerances, are to be expected. Thus, the inventive subject matter should not be construed as limited to the particular shapes of objects illustrated herein, but should include deviations in shapes that result, for example, from manufacturing. Thus, the objects illustrated in the figures are schematic in nature and their shapes are not intended to illustrate the actual shape of a region of a device and are not intended to limit the scope of the inventive subject matter.

The terminology used herein is for the purpose of describing particular embodiments only and is not intended to be limiting of the present inventive subject matter. As used herein, the singular forms “a,” “an” and “the” are intended to include the plural forms as well, unless the context clearly indicates otherwise. It will be further understood that the terms “comprises” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this present inventive subject matter belongs. It will be further understood that terms used herein should be interpreted as having a meaning that is consistent with their meaning in the context of this specification and the relevant art and will not be interpreted in an idealized or overly formal sense unless expressly so defined herein. The term “plurality” is used herein to refer to two or more of the referenced items. Although any methods, devices, and materials similar or equivalent to those described herein can be used in the practice or testing of the presently disclosed subject matter, representative methods, devices, and materials are now described.

In the drawings and specification, there have been disclosed typical preferred embodiments of the inventive subject matter and, although specific terms are employed, they are used in a generic and descriptive sense only and not for purposes of limitation, the scope of the inventive subject matter being set forth in the following claims.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A system for collecting, processing, and analyzing biometric data, the system comprising:
at least one sensor configured to be disposed on a measurement site on a user for measuring biosignals of the user;
a communication terminal communicatively coupled to the at least one sensor for receiving and transmitting the measured biosignals,
a computing device communicatively coupled to the communication terminal configured to receive transmitted biosignals from the communication terminal, the computing device comprising:
a memory;
one or more hardware processors configured for:
receiving the transmitted biosignals from the communication terminal;
processing the biosignals into a viewable representation of biometric data collected from the user, whereby the biometric data is compared to predefined parameters; and
generating a visual representation of the biometric data.

2. The system of claim 1, wherein the at least one sensor is selected from the group consisting of an electromyography sensor; an electrocardiography sensor; an electroencephalography sensor; a magnetomyography sensor; a mechanomyography sensor; a blood pressure sensor; a heart rate sensor; an accelerometer; and a gyroscope.

3. The system of claim 1, wherein the communication terminal further includes a microprocessor and analog-to-digital convertor chip; and
whereby firmware is flashed on the microprocessor to configure the analog-to-digital convertor chip to amplify the biosignals and convert the biosignals from analog to digital in a single chip.

4. The system of claim 1, whereby the at least one sensor is fixed within a textile-based material having a first surface and a second surface;
whereby the first surface is worn outward with respect to a user's body and the second surface is worn inward with respect to a user's body; and
whereby the at least one sensor is fixed within the textile-based material along the same plane of the textile-based material such that a contact surface of the at least one sensor is aligned in parallel with the second surface of the textile-based material such that the contact surface is substantially exposed to make contact with a user's skin.

5. The system of claim 4, whereby the textile-based material is substantially planar and fashioned into a circumferential band.

6. A system for collecting, processing, and analyzing biometric data, the system comprising:
at plurality of surface electromyography sensors configured to be disposed on a measurement site on a user for measuring biosignals of the user;
a communication terminal communicatively coupled to the plurality of surface electromyography sensors for receiving and transmitting the measured biosignals, such that the biosignals are sent separately in parallel;
a computing device communicatively coupled to the communication terminal configured to receive transmitted biosignals from the communication terminal, the computing device comprising:
a memory;
one or more hardware processors configured for:
receiving the transmitted biosignals from the communication terminal;
determining which of the plurality of surface electromyography sensors each biosignal is attributable to;
processing the biosignals into viewable representation of biometric data collected from the user, whereby the biometric data is compared to predefined parameters; and
generating a visual representation of the biometric data.

7. The system of claim 6, wherein the biometric data are collected within a time and frequency domain and are used to perform a plurality of calculations including intermuscular coherence, muscle activation ratio, co-contraction index, target-amplitude precision and accuracy, mean power frequency, average amplitude, signal envelope, data averaging, Fourier transformations, root mean square, signal burst, recruitment slope, peak frequency, and smoothed signal.

8. The system of claim 7, whereby the calculations are combined into a single representation of a user's progress in a rehabilitation process.

9. The system of claim 6, whereby the viewable representation of biometric data includes muscular rehabilitative exercises and suggested muscular movements that are updated in real time by the computing device based on the biosignals received from the communication terminal.

10. The system of claim 9, whereby the biosignals received from the communication terminal are collected within a time and frequency domain and are used to perform a plurality of calculations including intermuscular coherence, muscle activation ratio, co-contraction index, target-amplitude precision and accuracy, mean power frequency, average amplitude, signal envelope, data averaging, Fourier transformations, root mean square, signal burst, recruitment slope, peak frequency, and smoothed signal.

11. The system of claim 6, whereby the plurality of surface electromyography sensors is fixed within a textile-based material having a first surface and a second surface, whereby the first surface is worn outward with respect to a user and the second surface is worn inward with respect to a user; and
whereby the plurality of surface electromyography sensors is fixed within the textile-based material such that a contact surface of the plurality of surface electromyography sensors is aligned in parallel with the second surface of the textile-based material such that the contact surface is substantially exposed to make contact with a user's skin.

12. The system of claim 11, whereby the textile-based material is substantially planar and fashioned into a circumferential band for collecting electromyography biosignals.

13. A method of neuro-muscular rehabilitation comprising the steps of:
fixing at least one sensor within a textile-based material, wherein a contact surface of the sensor is exposed from the textile-based material;
placing the textile-based material on a user such that the contact surface of the sensor makes sufficient contact with a user's skin to collect and transmit biosignals;
transmitting the biosignals to a communication terminal for processing, whereby the biosignals are filtered to a frequency band in a range of 0.05 Hz to 20,000 Hz;
amplifying the biosignals and converting the biosignals from analog to digital in a single chip package;
transmitting the biosignals from the communication terminal to a computing device;
comparing biosignal data from the communication terminal to predefined parameters;
providing information to the user regarding muscular function with respect to predefined parameters; and
suggesting rehabilitative therapies to the user, whereby the rehabilitative therapies may be updated based on incoming biosignal data.
